(19) United States
(12) Patent Application Publication
(10) Pub. No.: US 2009/0005698 A1
(43) Pub. Date: Jan. 1, 2009

(54) Method and device for controlling motion module via brainwaves

(76) Inventors: Yu-Cheng Lin, Taipei Hsien (TW); Ryan Yeh, Taipei Hsien (TW); San-Chien Lin, Taipei Hsien (TW); Chang-Shiann Wu, Taipei Hsien (TW); Hsing-Chien Tsai, Taipei Hsien (TW); Shuo-Shian Wang, Taipei Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/819,764

(22) Filed: Jun. 29, 2007

(51) Int. Cl. A61B 5/0476

(52) U.S. Cl. 600/545

(57) ABSTRACT

A method for controlling motion module via brainwaves uses brainwaves to control and drive a motion module. The motion module can perform motions in a plurality of motion modes. The method for controlling motion module via brainwaves includes: firstly, obtaining a plurality of brainwave signals. Then, the characteristic value of each of the brainwave signals is analyzed. Next, the characteristic values of the brainwave signals are compared to determine which specified motion mode of the plurality of motion modes the motion module should move in. Finally, the motion module is driven to move in the specified motion mode.

### BACKGROUND OF THE INVENTION

1. Field of the Invention

The present invention relates to a method for controlling motion module via brainwaves. In particular, this invention relates to a method for controlling motion module via brainwaves and a device thereof that compares brainwave signals to control and drive a motion module.

2. Description of the Related Art

Brains dominate thought, activities, and movement of human beings. Brain science is an increasingly important area of moderate scientific research. Brainwave changes can be caused by internal events or externals event. Therefore, brainwaves truly represent a human being's physiology and psychology activities.

In order to improve the ability of a defective limb to take care of itself, a variety of medical equipment has been developed that is controlled by brainwaves, including artificial limbs, wheelchairs, and computers, etc. In the industrial control and household application research fields, brain science is combined with robotics. For example, the embedded Internet machine that is controlled by brainwaves of the prior art utilizes a human being's thought to control a remote robot to make the robot interact with its environment.

Neuro-linguistic programming is used for researching cognitive brain activities and shows that an external activity can be changed by controlling and converting a brain's thoughts. Thereby, learning and working can both be performed more efficiently. However, developed technology can currently only use brainwave signals to control equipment and cannot train one's brain activities to enhance the brain's ability.

### SUMMARY OF THE INVENTION

One particular aspect of the present invention is to provide a method for controlling motion module via brainwaves and a device thereof. It compares the characteristic values of a plurality of brainwave signals to control and drive a motion module to move by a motion mode. Thereby, brain activity is trained.

Another particular aspect of the present invention is to provide a method for controlling motion module via brainwaves and a device thereof. It uses the motion module to display the comparing result of the brainwave signals. The brain training activities are enjoyable.

The method for controlling motion module via brainwaves uses brainwaves to control and drive a motion module. The motion module can perform motions in a plurality of motion modes. The method for controlling motion module via brainwaves includes: firstly, obtaining a plurality of brainwave signals. The characteristic value of each of the brainwave signals is analyzed. Next, the characteristic values of the brainwave signals are compared to determine the motion module to move by a specified motion mode of the plurality of motion modes. Finally, the motion module is driven to move in the specified motion mode.

In a first embodiment, the characteristic value is the stability, the frequency, the activity, or the strength of the brainwave signal.

The brainwave controlling and driving device includes a motion module, a plurality of brainwave signal obtaining/processing modules, a computing module, and a motion mode determining/controlling module. The motion module can move in a plurality of motion modes. Each of the brainwave signal obtaining/processing modules obtains a brainwave signal, and amplifies, filters, and digitalizes the brainwave signal. The computing module is coupled with the brainwave signal obtaining/processing modules to receive the brainwave signals. The computing module analyzes the characteristic value for each brainwave signal and outputs the analyzed result. The motion mode determining/controlling module is coupled between the computing module and the motion module to receive the characteristic value of each of the brainwave signals. The motion mode determining/controlling module compares the characteristic values to control the motion module to move in the specified motion mode of the plurality of motion modes.

In another embodiment, the characteristic value is the stability, the frequency, the activity and the strength of the brainwave signal.

In another embodiment, the motion module includes a moving unit, and a driving unit. The moving unit can move along a plurality of directions. The driving unit is coupled with the motion mode determining/controlling module, and is controlled by the motion mode determining/controlling module to drive the moving unit to move along a specified direction of the directions.

In another embodiment, the brainwave signal obtaining/processing module includes a brainwave signal obtaining unit, a signal pre-processing unit, and an analog-to-digital converting unit. The brainwave signal-obtaining unit contacts the head of a living being to obtain a brainwave signal. The signal pre-processing unit is coupled with the brainwave signal-obtaining unit for amplifying and filtering the brainwave signal. The analog-to-digital converting unit is coupled with the signal pre-processing unit for digitalizing the brainwave signal.

In another embodiment, the computing module includes a plurality of computing units. The inputting terminal of each of the computing units is coupled with the brainwave signal obtaining/processing module for analyzing the characteristic value of the received brainwave signal and outputs the analyzed result to the motion mode determining/controlling module.

For further understanding of the invention, reference is made to the following detailed description illustrating the embodiments and examples of the invention. The description is only for illustrating the invention and is not intended to be considered limiting of the scope of the claim.

### BRIEF DESCRIPTION OF THE DRAWINGS

The drawings included herein provide a further understanding of the invention. A brief introduction of the drawings is as follows:

FIG. 1 is a schematic diagram of the system structure of the brainwave controlling and driving device of the present invention;

FIG. 2 is a schematic diagram of the system structure of the brainwave controlling and driving device of the first embodiment of the present invention;

FIG. 3 is a schematic diagram of the system structure of the brainwave controlling and driving device of the second embodiment of the present invention;

FIG. 4 is a schematic diagram of the appearance of the motion module in FIG. 3;

FIGS. 5a and 5b are schematic diagrams of the embodiment in FIG. 4 throwing a ball; and

FIG. 6 is a flow chart of the method for controlling motion module via brainwaves of the present invention.

### DETAILED DESCRIPTION OF THE PREFERRED EMBODIMENTS

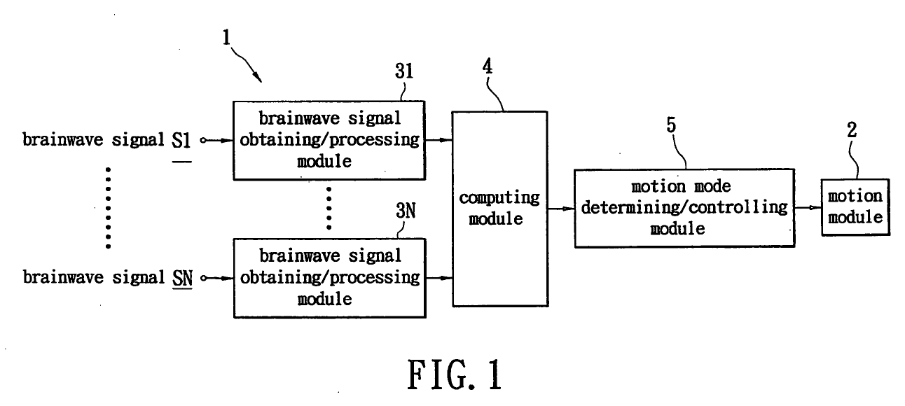

Reference is made to FIG. 1, which shows a schematic diagram of the system structure of the brainwave controlling and driving device of the present invention. The brainwave controlling and driving device 1 includes a motion module 2, a plurality of brainwave signal obtaining/processing modules 31˜3N, a computing module 4, and a motion mode determining/controlling module 5. N is an integer and is larger than one.

The motion module 2 can move in a plurality of motion modes. The brainwave signal obtaining/processing modules 31˜3N respectively obtain a brainwave signal S1˜SN, and amplify, filter, and digitalize the received brainwave signals S1˜SN. The computing module 4 is coupled with the brainwave signal obtaining/processing modules 31˜3N to receive the digitalized brainwave signals S1˜SN. The computing module 4 analyzes the characteristic value for each of the brainwave signals S1˜SN and outputs the analyzed result. The motion mode determining/controlling module 5 is coupled between the computing module 4 and the motion module 2 to receive the characteristic value of each of the brainwave signals S1˜SN. The motion mode determining/controlling module 5 compares the characteristic values of the brainwave signals S1˜SN to control the motion module 2 to move in the specified motion mode of the plurality of motion modes.

The characteristic value is the stability, the frequency, the activity and the strength of the brainwave signals S1˜SN. The motion module 2 includes a mechanical unit that can move in either one dimension or multiple dimensions. The motion mode can be a repeated movement of the mechanical unit in one axial or multiple axial directions, or a radial rotating motion in a clockwise or a counterclockwise direction. The motion mode determining/controlling module 5 is a comparing and controlling unit. The motion mode determining/controlling module 5 stores the data of the motion modes that corresponds to the brainwave signals S1˜SN. In details, the motion mode determining/controlling module 5 compares the characteristic values of the brainwave signals S1˜SN, such as the stability, the frequency, the activity and the strength of the brainwave signal, to determine which brainwave signal is dominating, and makes the motion module 2 move with a specified motion mode that corresponds to the brainwave signal. Therefore, the motion mode performed by the motion module 2 displays the compared result of the brainwaves S1˜SN.

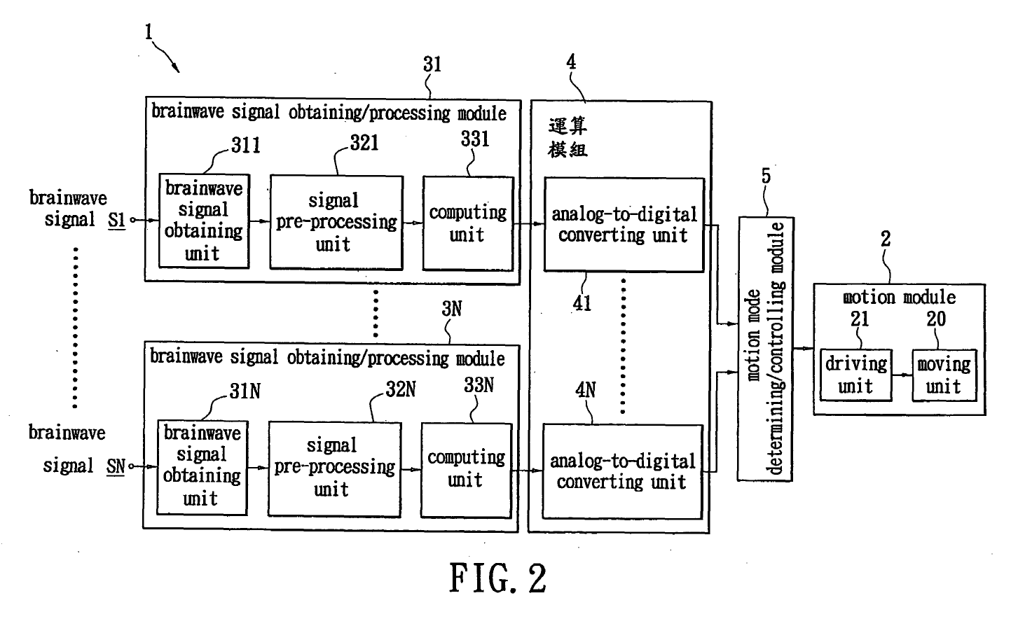

Reference is made to FIG. 2, which shows a schematic diagram of the system structure of the brainwave controlling and driving device of the first embodiment of the present invention. The brainwave signal obtaining/processing module 31 includes a brainwave signal-obtaining unit 311, a signal pre-processing unit 321, and an analog-to-digital converting unit 331. Similarly, the brainwave signal obtaining/processing module 3N includes a brainwave signal-obtaining unit 31N, a signal pre-processing unit 32N1, and an analog-to-digital converting unit 33N. The computing module 4 includes a plurality of computing units 41˜4N. The motion module 2 includes a moving unit 20, and a driving unit 21.

The brainwave signal obtaining units 311˜31N are composed of two or three pieces of electrodes which are pasted on a head of a living thing (generally meaning a human being) to obtain brainwave signals S1˜SN. The signal pre-processing units 321˜32N are respectively coupled with the brainwave signal obtaining units 311˜31N for receiving the brainwave signals S1˜SN. The signal pre-processing units 321˜32N amplify and filter the brainwave signals S1˜SN to obtain signals with a proper frequency. In order to achieve the above goals, the signal pre-processing units 321˜32N usually have a plurality of amplifiers and a plurality of filters with different frequency periods. The analog-to-digital converting units 331˜33N are respectively coupled with the signal pre-processing units 321˜32N for digitalizing the brainwave signals S1˜SN.

In FIG. 2, the computing module 4 includes a plurality of computing units 41˜4N. The inputting terminal of each of the computing units 41˜4N is coupled with each of the brainwave signal obtaining/processing modules 31˜3N. The computing units 41˜4N each include an integration computing circuit for analyzing the characteristic value of the received brainwave signal and output the analyzed result to the motion mode determining/controlling module 5. Next, the motion mode determining/controlling module 5 compares the analyzed results of the computing units 41˜4N to determine the motion mode for the motion module 2, and drives the motion module 2 to move according to the motion mode. A digital signal processing technology cooperates with the computing resource of the CPU of a computer to implement the calculating and controlling function of the computing module 4 and the motion mode determining/controlling module 5.

The motion module 2 includes a moving unit 20, and a driving unit 21. The driving unit 21 is coupled with the moving unit 20. The driving unit 21 is controlled and driven by the motion mode determining/controlling module 5 to drive the moving unit 20. In one embodiment, the moving unit 20 can move along a plurality of directions. When the moving unit 20 is moving along a specified direction of the directions it is called a motion mode. The driving unit 21 includes a motor that is driven by the motion mode determining/controlling module 5 to move the moving unit 20.

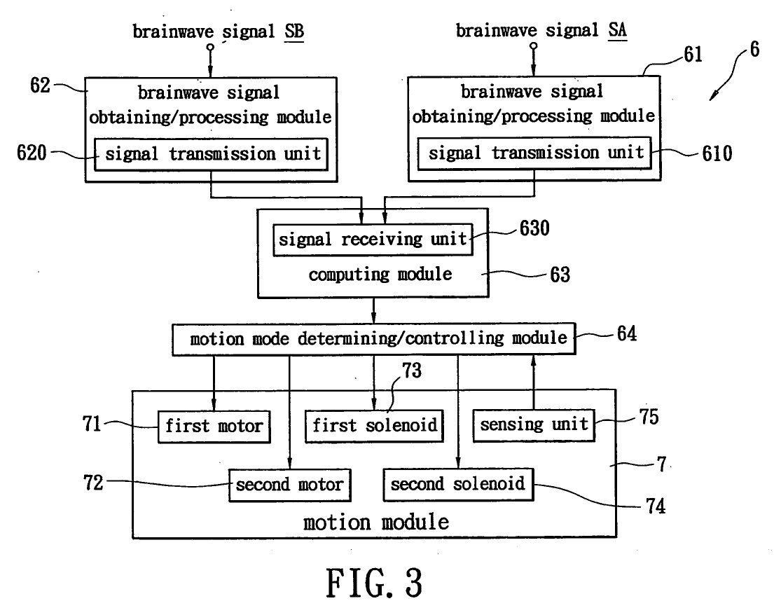

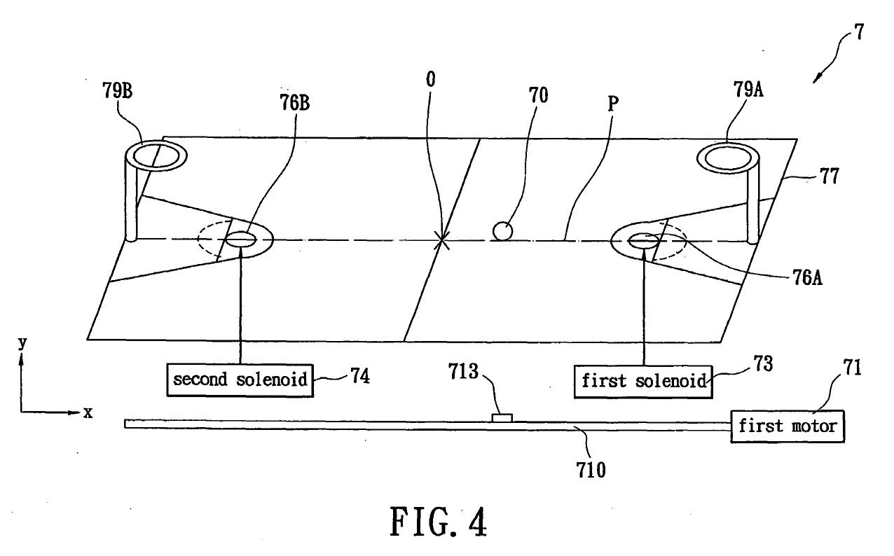

Reference is made to FIGS. 3 and 4. FIG. 3 is a schematic diagram of the system structure of the brainwave controlling and driving device 6 of the second embodiment of the present invention. FIG. 4 is a schematic diagram of the appearance of the motion module of the brainwave controlling and driving device 6 in FIG. 3. In this embodiment, the brainwave controlling and driving device 6 is a baseball practice device.

As shown in FIG. 3, the brainwave controlling and driving device 6 includes two brainwave signal obtaining/processing modules 61, 62, a computing module 63, a motion mode determining/controlling module 64, and a motion module 7. By referring to the system structure of FIG. 1, this embodiment compares the characteristic values of the two brainwave signals SA, SB to control and drive the motion module 7. The connecting relationship between each module and their operating principles will not be repeated again. The characteristic of this embodiment is that the brainwave signal obtaining/processing modules 61, 62 respectively have a signal transmission unit 610, 620, and the computing module 63 has a signal-receiving unit 630. The signal transmission units 610, 620 are coupled with the signal-receiving unit 630. The signal transmission units 610, 620 respectively transmit the brainwave signals SA, SB processed and digitalized by the brainwave signal obtaining/processing modules 61, 62 to the signal-receiving unit 630. Next, the computing module 63 compares the characteristic values of the brainwave signals SA, SB. In the second embodiment, the signal transmission units 610, 620 and the signal-receiving unit 630 use wireless communication technology to transmit the signals.

Another characteristic of this embodiment is that the motion module 7 uses the baseball device to display the compared result of the brainwave signals SA, SB. As shown in FIG. 3, the motion module 7 includes a first motor 71, a second motor 72, a first solenoid 73, a second solenoid 74, and a sensing unit 75. The motion mode determining/controlling module 64 drives the first motor 71, the second motor 72, the first solenoid 73 and the second solenoid 74. The sensing unit 75 includes a plurality of sensors (the sensors can be mechanical switches, electromagnetic switches, or photo sensors) for sensing the motion of the motion module 7 and generating and transmitting the sensing signal to the motion mode determining/controlling module 64. The motion mode determining/controlling module 64 drives the first motor 71, the second motor 72, the first solenoid 73 and the second solenoid 74 according to the sensing result of the sensing unit 75.

Reference is made to FIG. 3, which shows a schematic diagram of the appearance of the motion module 7. The motion module 7 includes a ball 70, and a base 77. The base 77 is similar to that of a baseball field. The base 77 has two basketball hoops 79A, 79B, and two linking ditches 76A, 76B. The basketball hoops 79A, 79B are located at two sides of the base 77, and the linking ditches 76A, 76B respectively correspond to the basketball hoops 79A, 79B and are located on the surface of the base 77. The first motor 71, the second motor 72 (not shown in the figure), the first solenoid 73, the second solenoid 74, and the sensing unit 75 are located below the base 77.

In FIG. 4, the base 77 further includes a sliding track 710, and a moving platform 713. The moving platform 713 is driven by the first motor 71, and it moves along the sliding track 710 in an x-direction or an inverse-x direction as the motor is rotating clockwise or counterclockwise. The moving platform 713 is magnetic so it can attract the ball 70. Therefore, the ball 70 is moved via the magnetic force of the moving platform 713 along the path P on the surface of the base 77. By referring to the motion mode of the ball 70 that is moving forwards in the x-direction (facing the basketball hoop 79A) or moving forwards in the inverse-x direction (facing the basketball hoop 79B), the compared result of the brainwave signals SA, SB are obtained.

In this embodiment, the location O is used as an origin of the coordinates and controls the ball 70 to move along the path P. In order to increase entertainment, the motion module 7 can shoot a ball. As shown in FIG. 4, the linking ditches 76A, 76B is the end point of the moving path for the ball 70. The first solenoid 73 and the second solenoid 74 are respectively located below the linking ditches 76A, 76B. When the ball 70 is moved to the end point of the moving path by the moving platform 713 and rolls into one of the linking ditches 76A, 76B, the sensing elements of the sensing unit 75 located around the linking ditches 76A, 76B are enabled to generate a sensing signal and the sensing signal is transmitted to the motion mode determining/controlling module 64. Thereby, the motion mode determining/controlling module 64 enables the solenoid (73 or 74) corresponding to the linking ditch (76A or 76B) to throw the ball 70 into the basketball hoop (79A or 79B).

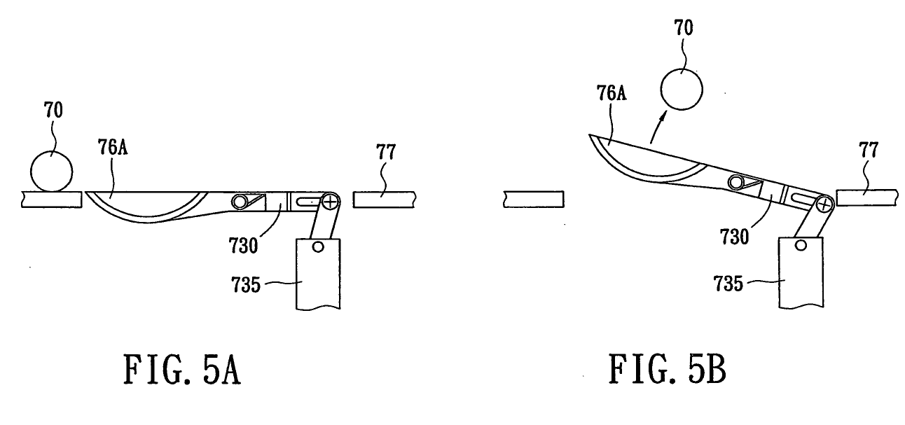

Reference is made to FIGS. 5A and 5B, which show schematic diagrams of a ball being thrown in this embodiment. The linking ditch 76A is connected with one end of a moving arm 730. A second end of the moving arm 730 is pivoted with a fastening rod 735. The moving arm 730 is controlled and driven by the first solenoid 73 and uses the location pivoted with the fastening rod 735 as a supporting point to rotate. When the ball 70 rolls into the linking ditch 76A along the surface of the base 77, the sensing element located around the linking ditch 76A is enabled to generate a sensing signal and the sensing signal is transmitted to the motion mode determining/controlling module 64. The motion mode determining/controlling module 64 responds to the motion of the ball rolling into the linking ditch 76A to drive the first solenoid 73 to rotate the moving arm 730. Thereby, the linking ditch 76A throws the ball 70 upwards and the ball 70 is thrown into the basketball hoop 79A along a parabolic curve path. Similarly, when the ball 70 rolls into the linking ditch 76B, the motion mode determining/controlling module 64 drives the second solenoid 74 to move the linking ditch 76A to throw the ball 70 into the basketball hoop 79B.

When the ball 70 returns to the surface of the base 77, the ball 70 comes back to below the location O along a channel (not shown in the figure) and the second motor 72 moves the ball 70 to the surface of the base 77. The sensing unit 75 further includes a plurality of location sensors disposed on the track 710 to detect the location of the moving platform 713. When the first motor 71 drives the moving platform 713 to the end point of the moving path, the location sensors detect this situation, and the motion mode determining/controlling module 64 stops the first motor 71 and drives the first motor 713 to rotate counterclockwise to move the moving platform 713 back to below the location O along the track 710. When the ball 70 comes back to the location O of the base 77, the ball 70 is attracted to the location O of the base.

Moreover, the brainwave controlling and driving device 6 further includes at least one attached module (not shown in the figure). The attached module is a lighting element or a screen that is controlled by the motion mode determining/controlling module 5 to display the operation station of the brainwave controlling and driving device 6, such as the compared result of the brainwave signals. Alternatively, it also displays the waveform of the brainwave signals.

The FIGS. 3˜5B and the related description uses a baseball machine as an example to describe the brainwave controlling and driving device 6. The brainwave controlling and driving device 6 can be another exercise practice device. This embodiment is used as an example, and it is not used to limit the scope of the present invention.

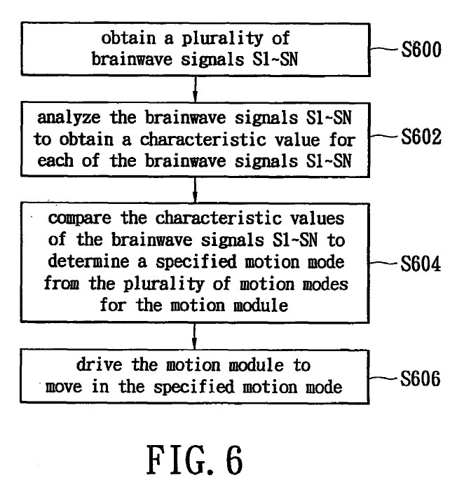

Reference is made to FIG. 6, which shows a flow chart of the method for controlling motion module via brainwaves of the present invention. The related system is shown in FIGS. 1 and 2. The method for controlling motion module via brainwaves is used for controlling and driving the motion module 2. The motion module 2 can perform a plurality of motions with different motion modes. The steps of the method for controlling motion module via brainwaves include:

Firstly, a plurality of brainwave signals S1˜SN are obtained (step S600).

Next, the brainwave signals S1˜SN are analyzed to obtain a characteristic value for each of the brainwave signals S1˜SN (step S602).

The characteristic values of the brainwave signals S1˜SN are compared to determine a specified motion mode from the plurality of motion modes for the motion module 2 (step S604).

Finally, the motion module 2 is driven to move in the specified motion mode (step S606).

The characteristic value is the stability, the frequency, the activity and the strength of the brainwave signals S1˜SN.

In another embodiment, after the step S600, a step of amplifying, filtering and digitizing the brainwave signals S1˜SN is included.

In another embodiment, the motion module 2 includes a moving unit 20. The moving unit 20 can move along a plurality of different directions. The specified motion mode means that the moving unit 20 moves along a specified direction of the plurality of different directions.

The brainwave controlling and driving device 6 compares the characteristic values of the brainwave signals, such as the stability, the frequency, the activity and the strength of the brainwave signals, to control the motion mode for the motion module. Thereby, the comparing result of the brainwave signals is displayed and the brain activity is trained. Furthermore, the brainwave controlling and driving device can be implemented into different exercise practice devices to increase the entertainment. Therefore, people will enjoy it.

The description above only illustrates specific embodiments and examples of the invention. The invention should therefore cover various modifications and variations made to the herein-described structure and operations of the invention, provided they fall within the scope of the invention as defined in the following appended claims.

What is claimed is:

1. A method for controlling motion module via brainwaves, used for controlling and driving a motion module via which the brainwave and the motion module can perform a plurality of motions with different motion modes, the steps comprising:
obtaining a plurality of brainwave signals;
analyzing the brainwave signals to obtain a characteristic value for each of the brainwave signals;
comparing the characteristic values of the brainwave signals to determine a specified motion mode from the plurality of motion modes for the motion module; and
driving the motion module to move in the specified motion mode.

2. The method for controlling motion module via brainwaves as claimed in claim 1, wherein the characteristic value is the stability, the frequency, the activity, and the strength of the brainwave signals.

3. The method for controlling motion module via brainwaves as claimed in claim 1, further comprising a step of amplifying, filtering, and digitizing the brainwave signals after the step of obtaining a plurality of brainwave signals;

4. The method for controlling motion module via brainwaves as claimed in claim 1, wherein the motion module includes a moving unit, and the moving unit moves along a plurality of different directions.

5. The method for controlling motion module via brainwaves as claimed in claim 4, wherein the specified motion mode means that the moving unit moves along a specified direction of the plurality of different directions.

6. A device for controlling motion module via brainwaves, comprising:
a motion module, wherein the motion module performs motions in a plurality of motion modes.
a plurality of brainwave signal obtaining/processing modules, wherein each of the brainwave signal obtaining/processing modules obtains a brainwave signal, and amplifies, filters, and digitalizes the brainwave signal;
a computing module coupled with the brainwave signal obtaining/processing modules to receive the brainwave signals, wherein the computing module analyzes the characteristic value for each brainwave signal and outputs the analyzed result; and
a motion mode determining/controlling module coupled between the computing module and the motion module to receive the characteristic value of each of the brainwave signals, wherein the motion mode determining/controlling module compares the characteristic values of the brainwave signals to control the motion module to move in the specified motion mode of the plurality of motion modes.

7. The device for controlling motion module via brainwaves as claimed in claim 6, wherein the characteristic value is the stability, the frequency, the activity and the strength of the brainwave signals.

8. The device for controlling motion module via brainwaves as claimed in claim 6, wherein the brainwave signal obtaining/processing modules comprises:
a brainwave signal-obtaining unit contacting a head of a living thing for obtaining the brainwave signal;
a signal pre-processing unit coupled with the brainwave signal-obtaining unit for amplifying and filtering the brainwave signal; and
an analog-to-digital converting unit coupled with the signal pre-processing unit for digitalizing the brainwave signal.

9. The device for controlling motion module via brainwaves as claimed in claim 6, wherein the computing module includes a plurality of computing units, an inputting terminal of each of the computing units is coupled with the brainwave signal obtaining/processing module for receiving and analyzing the characteristic value of the received brainwave signal and outputs the analyzed result to the motion mode determining/controlling module.

10. The device for controlling motion module via brainwaves as claimed in claim 6, wherein the brainwave signal obtaining/processing modules a signal transmission unit, the computing module includes a signal receiving unit, and the signal transmission unit is coupled with the signal receiving unit.

11. The device for controlling motion module via brainwaves as claimed in claim 10, wherein the signal transmission unit and the signal-receiving unit use wireless communication technology to link with the signal.

12. The device for controlling motion module via brainwaves as claimed in claim 6, wherein the motion module comprises:
a moving unit moving along a plurality of directions and
a driving unit coupled with the motion mode determining/controlling module and being controlled by the motion mode determining/controlling module to drive the moving unit to move along a specified direction of the plurality of directions.

13. The device for controlling motion module via brainwaves as claimed in claim 6, wherein the motion module includes a sensing unit, the sensing unit being coupled with the motion mode determining/controlling module, and the sensing unit detects the motion of the motion module and transmits the sensed result to the motion mode determining/controlling module.

14. The device for controlling motion module via brainwaves as claimed in claim 6, wherein the motion module comprises:
a track;
a motor controlled by the motion mode determining/controlling module; and
a moving platform located on the track, wherein the moving platform is connected with the motor and moves along the track as the motor rotates.

15. The device for controlling motion module via brainwaves as claimed in claim 14, wherein the motion module includes a ball and a base, the track, the motor and the moving platform are located below the base, and the moving platform attracts the ball via a magnetic force; thereby, the ball is moved on the base by the moving platform.

16. The device for controlling motion module via brainwaves as claimed in claim 14, wherein the motion module includes a plurality of location sensors, the location sensors are disposed on the track to detect the location of the moving platform and transmits the detected result to the motion mode determining/controlling module.

17. The device for controlling motion module via brainwaves as claimed in claim 14, wherein the motion module includes a solenoid, and the solenoid is controlled by the motion mode determining/controlling module.

18. The device for controlling motion module via brainwaves as claimed in claim 6, further comprising at least one attached module, and the attached module is controlled by the motion mode determining/controlling module.

19. The device for controlling motion module via brainwaves as claimed in claim 18, wherein the attached module is a lighting element or a screen.

20. The device for controlling motion module via brainwaves as claimed in claim 6, wherein the brainwave controlling and driving device is an exercise competition device.
